SEQUENCE LISTING (1 sequence)

SEQ ID NO: 1 - DNA, 996 bp, Bacillus subtilis
catatgtgcacaagtcttacattggaaactgctgaccgtaaacatgtattagcaagaacaatggattttgcatttcagctggggacagaggtgattctctatccgcgccgttacagctggaacagtgaagctgacggaagggcccatcagacacagtacgcgtttatcggtatggggagaaaacttggaaatatattatttgccgacggcattaatgaaagcggtttatcttgtgcggcgctttattttccgggctatgcggagtacgaaaaaacgatacgggaagataccgttcacattgtcccgcatgagtttgtgacatgggtgctgtcagtctgtcagtctttggaagacgtaaaagaaaagattcgatctttaacgattgtagagaaaaaattagatctattggatacagttttaccgcttcactggatattgtcagaccggacgggccgaaacctgacgatagaaccaagagcagacggcctcaaagtctatgataatcaacctggtgtcatgacgaacagccccgactttatatggcatgtaaccaatctgcagcaatatacaggaatcagaccgaagcaattagagagcaaagagatgggcggattagccctttctgcttttggccaaggcttaggaactgttggtctgccgggggattatacaccgccttcccggtttgtcagagctgtttatttgaaagaacatctagagccggcggccgatgaaacgaaaggtgtaacagccgcttttcaaattttggcaaatatgacgataccaaagggcgcagtgataacggaagaagacgaaattcattatacgcaatatacttccgtgatgtgcaacgaaactggaaactactatttccaccactatgacaatcggcaaatccaaaaagtaaatttatttcatgaagaccttgactgtttggagcctaaagtgttttccgctaaagcagaagagagtattcatgagcttaattaaggatcc

(19) United States
(12) Patent Application Publication
(10) Pub. No.: US 2005/0142652 A1
(43) Pub. Date: Jun. 30, 2005

(54) Process for production of large amount of penicillin V acylase

(75) Inventors: Hephzibah Sivaraman, Pune (IN); Archana Vishnu Pundle, Pune (IN); Cheravakkattu Gopalan Suresh, Pune (IN); George Guy Dodson, Yorkshire (GB); James Antony Brannigan, Yorkshire (GB)

Correspondence Address: FOLEY AND LARDNER;SUITE 500, 3000 K STREET NW, WASHINGTON, DC, 20007, US

(73) Assignees: COUNCIL OF SCIENTIFIC AND INDUSTRIAL RESEARCH; UNIVERSITY OF YORK

(21) Appl. No.: 10/812,387

(22) Filed: Mar. 30, 2004

Related U.S. Application Data
(63) Continuation of application No. PCT/IB03/06198, filed on Dec. 24, 2003.

(51) Int. Cl. C12N 9/16; C12N 9/80; C12N 1/21; C12N 15/74

(52) U.S. Cl. 435/196; 435/228; 435/69.1; 435/252.33; 435/488

(57) ABSTRACT

The present invention relates to a recombinant plasmid of FIG. 1, wherein (1) is pET-26b(+) cloning/expression region with SEQ ID No. 1 cloned between BamH I site 198 and Nde I site 288, (2) is lac I coding sequence, (3) is pBR322 origin, (4) is Kan coding sequence, and (5) is f1 origin; a recombinant E. Coli strain PTA 2456; and lastly, a process for the production of large amount of Penicillin V acylase using recombinant E. Coli strain PTA 2456.

### TECHNICAL FIELD

The present invention relates to a recombinant plasmid of FIG. 1, wherein (1) is pET-26b(+) cloning/expression region with SEQ ID No. 1 cloned between BamH I site 198 and Nde I site 288, (2) is lac I coding sequence, (3) is pBR322 origin, (4) is Kan coding sequence, and (5) is f1 origin. Also, it relates to a recombinant E. Coli strain PTA 2456. Further, it relates to a process for the production of large amount of Penicillin V acylase using recombinant E. Coli strain PTA 2456.

### BACKGROUND AND PRIOR ART REFERENCES OF THE PRESENT INVENTION

Penicillin V acylase (PVA) is an enzyme used for removing the phenoxyacetic acid group of benzyloxy penicillin (pen V) by hydrolysis to yield 6-aminopenicillanic acid (6-APA) which is used as a precursor in the commercial production of semi-synthetic penicillins. The strategy is similar to the production of 6-APA from benzyl penicillin (pen G) by employing penicillin G acylase (PGA). Penicillin acylase activity was discovered in many microorganisms including both bacteria and fungi as reported by Claridge et al., in “Bacterial Penicillin Amidase.” Nature 187, 237-238 (1960), Rolinson et al., “Formation of 6-Aminopenicillanic Acid from Penicillin by Enzymatic Hydrolysis.” Nature 187, 236-237 (1960), and Sakaguchi et al., “A Preliminary Report on a New Enzyme Penicillin Amidase,” J. Agr. Chem. Soc. Japan, 23, 411 (1950).

The inventors have been carrying out detailed studies on a Bacillus sphaericus penicillin acylase with penicillin V specificity. Gene for this enzyme has been cloned in E. coli (Olsson et al., “Molecular Cloning of Bacillus sphaericus Penicillin V Amidase Gene and Its Expression in Escherichia coli and Bacillus subtilis.” Appl. Environ. Microbiol. 49, 1084-1089 (1985)) and its gene sequence was determined and amino acid sequence of the protein deduced from gene sequence (Olsson & Uhlen, “Sequencing and heterologous expression of the gene encoding penicillin V amidase from Bacillus sphaericus.” Gene, 45, 175-181 (1986)). The structure of this enzyme has been solved at 2.5 Å by Suresh et al. “Penicillin V acylase crystal structure reveals new Ntn-hydrolase family members.” Nat. Struct. Biol. 6, 414-416 (1999). From the sequence data bank the reported sequence of conjugated bile acid hydrolase was found to have extensive homology with the sequence of B. sphaericus penicillin V acylase.

Importantly, the amino acid residues in the putative catalytic site of the solved structure of penicillin V acylase were found to be conserved in the reported sequence of bile acid hydrolase. The hydrolase produced by B. subtilis was picked up for further studies because of its similarity with penicillin V acylase. Observing the presence of the residues that are essential for the pen V acylase activity in hydrolase, it was intuitive that the enzyme has PVA activity. In order to test its activity, the hydrolase gene was cloned and expressed in E. coli. The result of the assay for the penicillin V acylase activity in the cells was positive and further the activity has been found to be much higher than that from other known bacillus sources.

### OBJECTS OF THE PRESENT INVENTION

The main object of the present invention is to develop a recombinant plasmid of FIG. 1, wherein (1) is pET-26b(+) cloning/expression region with SEQ ID No. 1 cloned between BamH I site 198 and Nde I site 288, (2) is lac I coding sequence, (3) is pBR322 origin, (4) is Kan coding sequence, and (5) is f1 origin.

Yet another main object of the present invention is to develop a recombinant E. Coli strain PTA 2456.

Still another object of the present invention is to develop a process for the production of large amount of Penicillin V acylase using recombinant E. Coli strain PTA 2456.

Still another object of the present invention is to develop a process, wherein the amount of Penicillin V acylase obtained in the recombinant stain is about 57 to 65 times more than in the ordinary conditions.

### SUMMARY OF THE PRESENT INVENTION

The present invention relates to a recombinant plasmid of FIG. 1, wherein (1) is pET-26b(+) cloning/expression region with SEQ ID No. 1 cloned between BamH I site 198 and Nde I site 288, (2) is lac I coding sequence, (3) is pBR322 origin, (4) is Kan coding sequence, and (5) is f1 origin; a recombinant E. Coli strain PTA 2456; and lastly, a process for the production of large amount of Penicillin V acylase using recombinant E. Coli strain PTA 2456.

### DETAILED DESCRIPTION OF THE PRESENT INVENTION

Accordingly, the present invention relates to a recombinant plasmid of FIG. 1, wherein (1) is pET-26b(+) cloning/expression region with SEQ ID No. 1 cloned between BamH I site 198 and Nde I site 288, (2) is lac I coding sequence, (3) is pBR322 origin, (4) is Kan coding sequence, and (5) is f1 origin; a recombinant E. Coli strain PTA 2456; and lastly, a process for the production of large amount of Penicillin V acylase using recombinant E. Coli strain PTA 2456.

In still another embodiment of the present invention, wherein a recombinant plasmid of FIG. 1, wherein (1) is pET-26b(+) cloning/expression region with SEQ ID No. 1 cloned between BamH I site 198 and Nde I site 288, (2) is lac I coding sequence, (3) is pBR322 origin, (4) is Kan coding sequence, and (5) is f1 origin.

In still another embodiment of the present invention, wherein the SEQ ID No. 1 is the sequence of Bacillus subtilis gene of FIG. 2, encoding conjugated bile acid hydrolase.

In still another embodiment of the present invention, wherein a recombinant E. Coli strain PTA 2456.

In still another embodiment of the present invention, wherein the recombinant stain produces an amount of Penicillin V acylase about 57 to 65 times more than in the ordinary conditions.

In still another embodiment of the present invention, wherein the strain comprises recombinant plasmid of FIG. 1, whereby (1) is pET-26b(+) cloning/expression region with SEQ ID No. 1 cloned between BamH I site 198 and Nde I site 288, (2) is lac I coding sequence, (3) is pBR322 origin, (4) is Kan coding sequence, and (5) is f1 origin.

In still another embodiment of the present invention, wherein a process for the production of large amount of Penicillin V acylase using recombinant E. Coli strain PTA 2456, said process comprising steps of:
preparing a recombinant plasmid of FIG. 1, wherein (1) is pET-26b(+) cloning/expression region with SEQ ID No. 1 cloned between BamH I site 198 and Nde I site 288, (2) is lac I coding sequence, (3) is pBR322 origin, (4) is Kan coding sequence, and (5) is f1 origin,
transforming the competent cells of E. coli with the recombinant plasmid to obtain recombinant strain PTA 2456,
growing the strain in a fermentation medium for time period ranging between 4 to 18 hours at temperature ranging between 30 to 40° C., and
obtaining the large amount of Penicillin V acylase.

In still another embodiment of the present invention, wherein the amount of Penicillin V acylase obtained in the recombinant stain is about 57 to 65 times more than in the ordinary conditions.

In still another embodiment of the present invention, wherein the fermentation medium comprises bacto-tryptone of concentration ranging between 8-10 g/l, bacto-yeast extract of concentration ranging between 5-8 g/l, sodium chloride of concentration ranging between 3-5, and an antibiotic of concentration ranging between 30-50 μg/ml.

In still another embodiment of the present invention, wherein the E. coli strain is BL-21 DE3.

In still another embodiment of the present invention, wherein described herein is the cloning of the gene producing the said enzyme from Bacillus subtilis NCIMB11621 onto Escherichia coli using a suitable plasmid vector to obtain a recombinant organism and its use in order to achieve an increased production of penicillin V acylase. The activity of the recombinant organism is similar to the activity of an enzyme hitherto described as conjugated bile acid hydrolase, is shown to yield an increased production of penicillin V acylase. The B. subtilis enzyme responsible for the penicillin V acylase activity has been discovered after the structure solution of Bacillus sphaericus penicillin V acylase and on comparison of the gene sequence of B. subtilis, reported to be that of conjugated bile acid hydrolase (CBH), with that of Penicillin V acylase gene of B. sphaericus. The comparison resulted in the identification of amino acid residues responsible for penicillin V acylase activity being conserved between the two gene sequences. The penicillin V acylase activity was subsequently confirmed using purified enzyme preparation as well.

The present invention relates to a Bacillus subtilis gene cloned in Escherichia coli and a process for the production of penicillin V acylase using the said clone. More particularly it relates to a method for increasing the production of penicillin V acylase by cloning in E. coli, a gene from B. subtilis whose product is known as conjugated bile acid hydrolase, but which also showed penicillin V acylase activity.

### BRIEF DESCRIPTION OF THE ACCOMPANYING DRAWINGS

FIG. 1 shows recombinant vector produced in accordance with the present invention wherein (1) is pET-26b(+)cloning/expression region and nucleotide sequence described in FIG. 2 is cloned between BamH I (198) and Nde I(288), (2) is lac I coding sequence, (3) is pBR322 origin, (4) is Kan coding sequence & (5) is f1 origin

FIG. 2 shows complete nucleotide of Bacillus subtilis gene encoding conjugated bile acid hydrolase inserted between sites of restriction enzymes Bam H I and Nde I of cloning/expression region (1), in FIG. 1.

The present invention also provides a process for the preparation of Penicillin V acylase using the said recombinant PTA 2456, which comprises of growing the said PTA 2456 in a conventional fermentor medium for a period of 4 to 18 hrs. at a temperature ranging between 30 to 40° C., separating the biomass and recovering the product by conventional methods used for separation of wild type enzyme.

In one of the embodiments of the present invention the conventional fermentation medium consists of following composition (g/l): bacto-tryptone, 8-10; bacto-yeast extract, 5-8; sodium chloride, 3-5 and an antibiotic 30-50 μg/ml.

In a feature of the present invention the product is separated by conventional methods such as sonication of microbial cell mass, ammonium sulphate fractionation of the sonicate followed by hydrophobic interaction chromatography.

In a feature of the present invention a method is described wherein DNA recombinant technology has been exploited to achieve an improved production of penicillin V acylase (PVA). The yields obtained using the new strain are substantially higher than those reported so far. The method of invention comprises of isolating a selected chromosomal fragment from B. subtilis NCIMB 11621 containing the gene encoding the reported enzyme identified as conjugated bile acid hydrolase (CBH), incorporating this DNA fragment into a multi copy vector and subsequently transforming the competent cells of E. coli (BL-21 DE3) using the modified plasmid.

In another feature of the present invention a method of cloning a B. subtilis hydrolase enzyme into E. Coli. A selected 1 kb chromosomal DNA fragment from B. subtilis NCIMB 11621, which represented the gene encoding the enzyme originally known as conjugated bile acid hydrolase and having PVA activity, as observed by the inventors of the present invention, has been inserted into the vector plasmid and then the modified plasmid was introduced into E. coli.

Also, in accordance with the present invention there is provided a new strain of E. coli namely (PTA 2456) having enhanced penicillin V acylase productivity which includes within it a cloning vector pET-26b containing an insert of a 1 kb chromosomal DNA fragment of B. subtilis NCIMB11621.

The multi copy plasmid construct used for the transformation of E. coli cells has been designed from the vector pET-26b by inserting a fragment of about 1 kb DNA drawn from B. subtilis NCIMB 11621 in the cloning region between the sites of restriction enzymes BamH I and Nde I.

The chromosomal DNA fragment, 1-10 kb length, of B. subtilis that include the appropriate gene coding the enzyme responsible for conjugated bile acid hydrolase activity, could be inserted into the pET 26b vector, following the procedure described by Sambrook et al., Molecular Cloning, A Laboratory Manual, Vol.1, p182 (1988) to form an appropriate plasmid. The plasmid thus formed may be then cloned in E. coli using the procedure described therein.

The following microorganisms are available from the permanent collection of the “American Type Culture Collection”, 12301, Parklawn Drive, Rockville, Md. 20852.

| | |
|---|---|
| Bacillus sphaericus | NCIM 2478, ATCC 14577 |
| Bacillus subtilis | NCIMB 11621, BGSC 1A436 |
| Escherichia coli | ATCC for deposit accession number: PTA 2456 |
| (a recombinant | |
| with a plasmid | |
| construct therein) | |

The instant invention is further elaborated with the help of examples. However, they should not be construed to limit the scope of the invention.

### EXAMPLE 1

A plasmid containing a chromosomal DNA fragment from B. subtilis NCIMB 11621 was prepared as follows:

The vector pET-26b(+) 5360 bp used for the cloning of conjugated bile acid hydrolase gene in E. coli. The vector has two restriction enzyme sites, one for Nde I and another for BamH I, and it also confers resistance towards kanamycin. These characteristics were made use for inserting the conjugated bile acid hydrolase gene and testing the expression of the vector.

The reference conjugated bile acid hydrolase gene was amplified through PCR using chromosomal DNA of B. subtilis as template along with upstream and downstream primers. The vectors as well as the amplified insert were subjected to the restriction enzyme digestion by Nde I and BamH I, followed by ligation using T4 DNA ligase (Sambrook et al , Molecular Cloning, A Laboratory Manual, Vol.1, p182 (1988)). The construct thus obtained was used for the transformation of competent cells of E. coli BL-21(DE3) in order to achieve the expression of the desired protein (Sambrook et al Molecular Cloning, A Laboratory Manual, Vol.1, p182 (1988)). The transformed cells were selected by picking up colonies grown on Luria-Bertani agar medium containing kanamycin. To further confirm the results, the plasmid was isolated from the transformants and digested with Nde I and BamH I. The digestion product was subjected to electrophoresis on 1% Agarose gel which showed bands corresponding to the size of the plasmid and that corresponding to the size (1 kb) of the insert.

### EXAMPLE 2

The resultant transformed E. coli PTA 2456 with plasmid containing conjugated bile acid hydrolase gene within was tested for penicillin V acylase production.

The 500 ml Erlenmyer flasks containing 100 ml LB medium were inoculated aseptically using 1 ml each of the overnight grown culture of the said clone. The flasks were incubated at 37° C. and 150 rpm. Isopropyl β-D Thiogalactopyranoside (IPTG) was added aseptically when the culture reached OD_{595 λ} value between 0.2-0.6. The incubation was continued for next 3-4 hrs and the cells were harvested by centrifugation. The penicillin V acylase activity was checked using whole cells and cell-free extracts. The crude extract was incubated with 2% potassium salt of penicillin V in citrate buffer of 0.1 molarity and pH 5.8 at 40° C. for 10 minutes. The 6-aminopenicillanic acid (6-APA) formed was estimated using PDAB by the method of Bomstein & Evans “Automated calorimetric determination of 6-aminopenicillanic acid in fermentation”. Anal. Chem., 37, 576-578 (1965) as modified by Shewale et al, “Evaluation of determination of 6-aminopenicillanic acid by p-methyl-aminobenzaldehyde”, Biotechnol. Tech. 1, 69-72 (1987).

The results are set out in the table:

TABLE-1

| Organism/clone | U/g/h wet cells | IU/g dry cells |
|---|---|---|
| B. sphaericus (NCIM 2478) | 165 | 3.57 |
| Conjugated bile acid hydrolase- | 8799 | 203.68 |
| clone PTA 2456 | | |

The results indicate that penicillin V acylase yield of the clone is almost 50 times higher than that of wild type strain under identical condition.

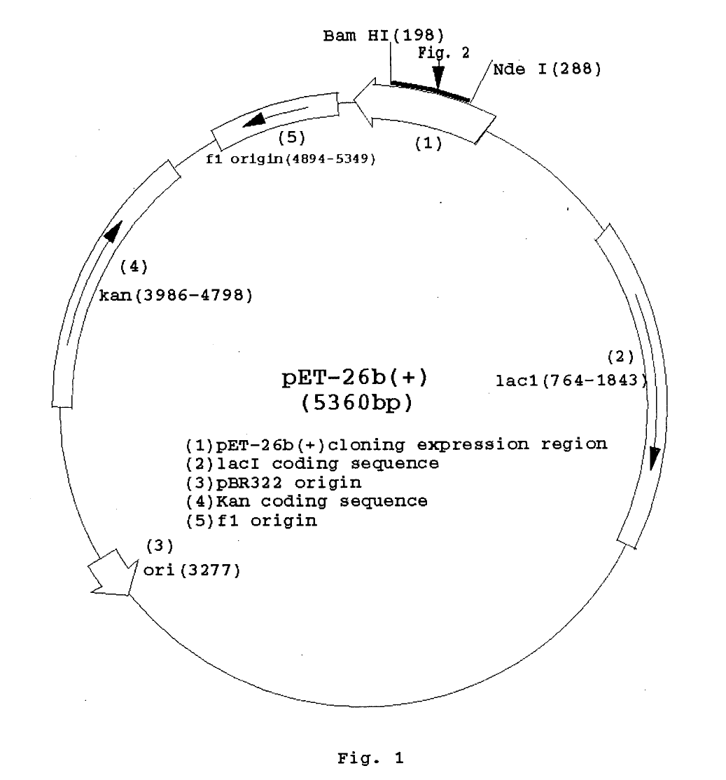

1. A recombinant plasmid of FIG. 1, wherein (1) is pET-26b(+) cloning/expression region with SEQ ID No. 1 cloned between BamH I site 198 and Nde I site 288, (2) is lac I coding sequence, (3) is pBR322 origin, (4) is Kan coding sequence, and (5) is f1 origin.

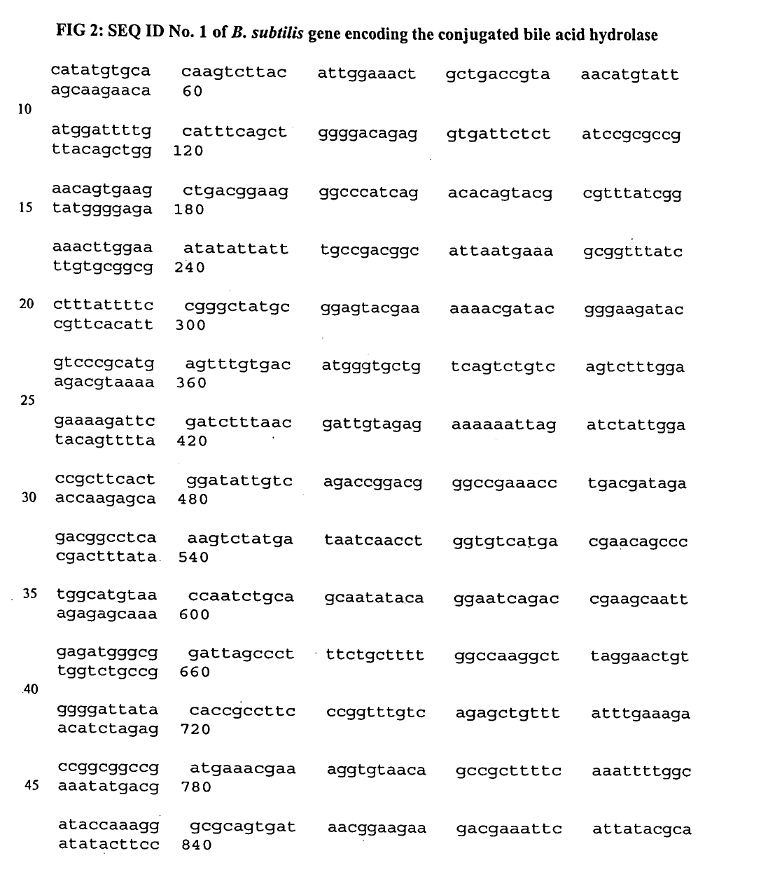

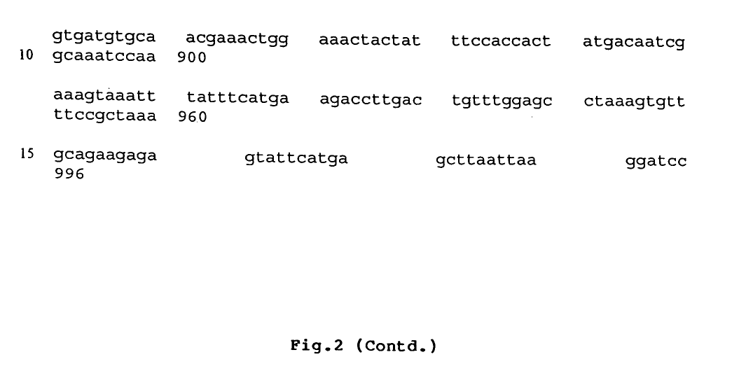

2. A recombinant plasmid as claimed in claim 1, wherein the SEQ ID No. 1 is the sequence of Bacillus subtilis gene of FIG. 2, encoding conjugated bile acid hydrolase.

3. A recombinant E. Coli strain PTA 2456.

4. A recombinant strain as claimed in claim 3, wherein the recombinant stain produces an amount of Penicillin V acylase about 57 to 65 times more than in the ordinary conditions.

5. A recombinant strain as claimed in claim 3, wherein the strain comprises recombinant plasmid of FIG. 1, whereby (1) is pET-26b(+) cloning/expression region with SEQ ID No. 1 cloned between BamH I site 198 and Nde I site 288, (2) is lac I coding sequence, (3) is pBR322 origin, (4) is Kan coding sequence, and (5) is f1 origin.

6. A process for the production of large amount of Penicillin V acylase using recombinant E. Coli strain PTA 2456, said process comprising steps of:
a. preparing a recombinant plasmid of FIG. 1, wherein (1) is pET-26b(+) cloning/expression region with SEQ ID No. 1 cloned between BamH I site 198 and Nde I site 288, (2) is lac I coding sequence, (3) is pBR322 origin, (4) is Kan coding sequence, and (5) is f1 origin,
b. transforming the competent cells of E. coli with the recombinant plasmid to obtain recombinant strain PTA 2456,
c. growing the strain in a fermentation medium for time period ranging between 4 to 18 hours at temperature ranging between 30 to 40° C., and
d. obtaining the large amount of Penicillin V acylase.

7. A process as claimed in claim 6, wherein the amount of Penicillin V acylase obtained in the recombinant stain is about 57 to 65 times more than in the ordinary conditions.

8. A process as claimed in claim 6, wherein the fermentation medium comprises bacto-tryptone of concentration ranging between 8-10 g/l, bacto-yeast extract of concentration ranging between 5-8 g/l, sodium chloride of concentration ranging between 3-5, and an antibiotic of concentration ranging between 30-50 μg/ml.

9. A process as claimed in claim 6, wherein the E. coli strain is BL-21 DE3.
